(19) United States
(12) Patent Application Publication
(10) Pub. No.: US 2021/0116408 A1
(43) Pub. Date: Apr. 22, 2021

(54) Improved Electrode for Electrochemical Device

(71) Applicant: AegirBio AB, Lund (SE)

(72) Inventors: Sudha SRIVASTAVA, New Delhi (IN); Rahul SAXENA, Uttarakhand (IN)

(21) Appl. No.: 17/041,643

(22) PCT Filed: Mar. 25, 2019
(86) PCT No.: PCT/IB2019/052388

§ 371 Date: Sep. 25, 2020

(30) Foreign Application Priority Data
Mar. 29, 2018 (IN) 201811012008

(51) Int. Cl. G01N 27/327; G01N 33/543; G01N 33/78

(52) U.S. Cl. CPC G01N 27/3275; G01N 2333/59; G01N 33/78; G01N 33/5438

(57) ABSTRACT

The present disclosure is on a premise that the inventors of the present disclosure surprisingly observed that an electrode attached with graphene-polypyrrole based nano-composites can significantly improve the conductivity of the electrode, which in turn can significantly improve limit of detection (LOD) of the electrochemical device enabling quantitative detection of biological target in a sample to the tune of 0.5 fg/mL. Accordingly, an aspect of the present disclosure relates to an improved electrode for an electrochemical device, the electrochemical device capable of detecting a biological target in a sample, wherein at least part of a surface of the electrode is attached with a graphene-polypyrrole based composite, and wherein the graphene-polypyrrole based composite is attached with at least one biological targeting moiety. Aspects of the present disclosure also provide a method of the fabrication of the advantageous electrode of the present invention, an electrochemical device including the advantageous electrode and method of detection of a biological target.

### FIELD OF THE INVENTION

The present disclosure pertains to an improved electrode for an electrochemical device. In particular, the present disclosure provides an improved electrode for electrochemical device enabling detection of a biological target in a sample. Aspect of the present disclosure also provides an electrochemical device for detection of a biological target in a sample.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Diagnostic instruments has evolved over the last five decades with single indirect assay available till 1950s to a multitude of instruments/techniques like radioimmunoassay (RIA), Enzyme Linked Immuno Sorbent Assay (ELISA), Flourescence based (FIA), Chemiluminescence based (CLIA) and bioluminescence based immunosorbent assays. Thyroid hormones in healthy individuals range between 2.3-4.2 pg/mL (free T3), 0.8-2.0 ng/ml (total T3), 0.008-0.018 ng/mL (free T4), 0.045-0.125 μg/mL (total T4) and 0.3-3.04 μIU/mL (TSH). As per the recommendations of The National Academy of Clinical Biochemistry (NACB) the minimum detectable concentration (LOD) of TSH assay should be less than or equal to 0.02 mIU/L. This permits patients with non-thyroid illness to be distinguished from those with primary hyperthyroidism.

The RIA based assays have high sensitivity and detection range (T3: 0.08-8 ng/mL, T4: 0.11-2.49 ng/mL, TSH: 0.1-90 μIU/mL). However, radioisotope associated radiation hazards limits its usage. On the other hand, ELISA being safe and cost effective captured more than 90% of the diagnostic market despite having comparatively poorer detection range (T3: 0.2-10 ng/mL, T4: 0.044-0.108 ug/mL, TSH: 0.2-40 μIU/mL). Currently, most laboratories measure T4 and T3 concentrations by competitive immunometric assays performed on automated platforms using enzymes, fluorescence or chemiluminescent molecules as signals. The sensitivity and detection range of CLIA is comparable to that of RIA (T3: 0.02-7.5 ng/mL, T4: 0.001-0.25 ug/mL, TSH: 0.2-100 μIU/mL) at the same time no radiation hazards and automated assay procedure is the cause of its wide popularity. Still CLIA could not take over the market of ELISA based assays due to high capital cost of CLIA instrument.

These methods despite being highly sensitive, require transportation of samples to laboratories, trained manpower, and are time consuming. The cost and portability issues have been well addressed by Point-of-Care (POC) device employing lateral flow immuchromatographic assays (LFA) developed for Semi-quantitative estimation of TSH for hypothyroidism serum samples (above 5 μIU/mL). However, for normal range or hyperthyroidism serum samples LFA could not be applied. Last five years witnessed a major shift in performance in LFA devices with cell phone interface readout system improving detection limits for TSH as low as 0.31 μIU/mL (You et al; Biosensors & Bioelectronics; vol 40, 180-185). The reproducibility of LFIA test is compromised due to variations in membrane batches, temperature, humidity, heat, air, and sunlight. In addition, in many test formats pre-treatment of samples becomes mandatory where significant interferents are present. Above all, restrictions in the limit of detection of these platforms restrict their use to the determination of analytes that are highly abundant in the sample tested (TSH) while no LFA is available for T3 and T4, probably due to clinically relevant lower concentrations.

These shortcomings of LFA based POCs can be resolved by electrochemical biosensors holding great promise as a platform for POC owing to its advantages such as sensitivity, rapidity, simplicity, inexpensive cost and portability. Electrochemical immunosensors employing Interdigitated electrodes and sandwich immunoassay format presented an LOD of 0.012 μIU/mL for TSH as opposed to 0.1 μIU/mL and 0.2 μIU/mL for RIA and CLIA based kits. Third generation electrochemiluminescence assay (ECLIA) Elecsys 2010 could achieve LOD of 0.005 μIU/mL (Kazerouni et al; Caspian J Intern Med., 2012 Spring; 3(2): 400-104).

The published US patent document (US20150247816) discloses an electrochemical biosensor comprising: a) a sensing electrode having attached to its surface a binding agent capable of specifically binding to the analyte to form a binding agent-analyte complex and wherein the binding of the analyte to the binding agent alters the electron transfer properties at the sensing electrode surface thereby providing a change in the electrochemical response at the sensing electrode surface proportional to the number of binding agent-analyte complexes, and b) a test equipment capable of measuring the electrochemical response at the sensing electrode surface. However, the disclosed biosensor exhibits the limit of detection (LOD) of 10 pg/mL.

Accordingly, there remains a need of an improved electrode that can improve the sensitivity and specificity of the electrochemical device. Particularly, need is felt of an electrode that can enable an electrochemical device to detect the biomolecule (biological target) present in a femtogram scale in the sample. The present disclosure fulfils the existing needs, inter-alia, others and provides an improved electrode and an electrochemical device including the improved electrode.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### OBJECTS OF THE INVENTION

It is an object of the present disclosure to provide an improved electrode for an electrochemical device.

It is another object of the present disclosure to provide an improved electrode for an electrochemical device capable of detecting a biological target in a sample.

It is another object of the present disclosure to provide an electrochemical device that can detect the biomolecule (biological target) present on a femtogram scale in the sample.

It is another object of the present disclosure to provide an electrochemical device for detection of thyroid hormone(s).

It is another object of the present disclosure to provide an electrochemical device for quantitative detection of thyroid hormone(s).

Still further object of the present disclosure is to provide a method of fabrication of an improved electrode for an electrochemical device.

Still further object of the present disclosure is to provide a method of fabrication of an electrochemical device for detection of a biomolecule (biological target) in the sample.

Still further object of the present disclosure is to provide a method of quantitative detection of a biomolecule (biological target) in the sample.

Still further object of the present disclosure is to provide a method of quantitative detection of any or a combination of thyroxine (T4), triiodothyronine (T3) and thyroid stimulating hormone (TSH) in a sample.

### SUMMARY

The present disclosure pertains to an improved electrode for an electrochemical device. In particular, the present disclosure provides an improved electrode for electrochemical device enabling detection of a biological target in a sample. Aspect of the present disclosure also provides an electrochemical device for detection of a biological target in a sample.

An aspect of the present disclosure provides an improved electrode for an electrochemical device, the electrochemical device capable of detecting a biological target in a sample, wherein at least part of a surface of the electrode is attached with a graphene-polypyrrole based composite, and wherein the graphene-polypyrrole based composite is attached with at least one biological targeting moiety. In an embodiment, the biological target is selected from any or a combination of an antibody, an antibody derivative, a hapten and an antigen. In an embodiment, the biological target is selected from any or a combination of a hormone, a protein, a polysaccharide, a lipid, a polynucleotide, and a metabolite. In an embodiment, the biological target is selected from any or a combination of thyroxine (T4), triiodothyronine (T3) and thyroid stimulating hormone (TSH). In an embodiment, the graphene-polypyrrole based composite comprises graphene-polypyrrole based nano-composite. In an embodiment, the at least part of the surface of the electrode is coated with the graphene-polypyrrole based composite. In an embodiment, the at least part of the surface of the electrode is functionalized with one or more amino groups capable of forming covalent bond with the graphene-polypyrrole based composite. In an embodiment, the at least one biological targeting moiety comprises one or a plurality of agents capable of selectively capturing the biological target. In an embodiment, the at least one biological targeting moiety comprises one or a plurality of agents capable of non-selectively capturing the biological target. In an embodiment, the at least one biological targeting moiety is selected from any or a combination of an anti-T3 antibody, an anti-T4 antibody and an anti-TSH antibody. In an embodiment, the graphene-polypyrrole based composite is attached with the at least one biological targeting moiety through an amide linkage. In an embodiment, the graphene-polypyrrole based composite is functionalized with one or more amino groups capable of forming the amide linkage with Fc region of any of the anti-T3 antibody, the anti-T4 antibody and the anti-TSH antibody.

Another aspect of the present disclosure provides an electrochemical device for detection of a biological target in a sample, the electrochemical device comprising at least one electrode defining a surface, wherein at least a part of the surface of the electrode is attached with a graphene-polypyrrole based composite, and wherein the graphene-polypyrrole based composite is attached with at least one biological targeting moiety. In an embodiment, the biological target is selected from any or a combination of an antibody, an antibody derivative, a hapten and an antigen. In an embodiment, the biological target is selected from any or a combination of a hormone, a protein, a polysaccharide, a lipid, a polynucleotide, and a metabolite. In an embodiment, the biological target is selected from any or a combination of thyroxine (T4), triiodothyronine (T3) and thyroid stimulating hormone (TSH). In an embodiment, the graphene-polypyrrole based composite comprises graphene-polypyrrole based nano-composite. In an embodiment, the at least part of the surface of the electrode is coated with the graphene-polypyrrole based composite. In an embodiment, the at least part of the surface of the electrode is functionalized with one or more amino groups capable of forming covalent bond with the graphene-polypyrrole based composite. In an embodiment, the at least one biological targeting moiety comprises one or a plurality of agents capable of selectively capturing the biological target. In an embodiment, the at least one biological targeting moiety comprises one or a plurality of agents capable of non-selectively capturing the biological target. In an embodiment, the at least one biological targeting moiety is selected from any or a combination of an anti-T3 antibody, an anti-T4 antibody and an anti-TSH antibody. In an embodiment, the graphene-polypyrrole based composite is attached with the at least one biological targeting moiety through an amide linkage. In an embodiment, the graphene-polypyrrole based composite is functionalized with one or more amino groups capable of forming the amide linkage with Fc region of any of the anti-T3 antibody, the anti-T4 antibody and the anti-TSH antibody. In an embodiment, the at least one electrode is a sensing electrode. In an embodiment, the electrochemical device exhibits the limit of detection (LOD) of 0.001 μIU/mL, 0.5 fg/mL and 0.5 fM for thyroid stimulating hormone (TSH), thyroxine (T4) and triiodothyronine (T3), respectively. In an embodiment, the electrochemical device effects quantitative detection of any of a combination of the thyroid stimulating hormone (TSH), the thyroxine (T4) and the triiodothyronine (T3) within 20 minutes.

Still further aspect of the present disclosure relates to a method of fabrication of a working electrode for an electrochemical device, the method comprising the steps of: taking a working electrode; treating the working electrode with an agent capable of functionalizing at least a part of the surface of the working electrode to form a functionalized working electrode; incubating the functionalized working electrode with graphene-polypyrrole composite or nanocomposite to form a surface modified working electrode; treating the surface modified working electrode with an agent capable of functionalizing at least a part of the surface of the graphene-polypyrrole composite; and attaching at least one biological targeting moiety with the graphene-polypyrrole composite to realize the working electrode for the electrochemical device.

Various objects, features, aspects and advantages of the inventive subject matter will become more apparent from the following detailed description of preferred embodiments.

### BRIEF DESCRIPTION OF DRAWINGS

FIG. 1 illustrates an exemplary diagram depicting an improved electrode realized in accordance with embodiments of the present disclosure.

FIG. 2 illustrates an exemplary diagram depicting an electrochemical device for detection of a biological target in a sample, in accordance with an embodiment of the present disclosure.

FIG. 3 illustrates an exemplary diagram depicting an electrochemical device for detection of a biological target in a sample including an improved electrode, realized in accordance with an embodiment of the present disclosure.

FIGS. 4A and 4B illustrate exemplary TSH quantification curve and corresponding calibration plot using Electrochemical Impedance Spectroscopy (EIS), in accordance with the embodiments of the present disclosure.

FIGS. 5A and 5B illustrate exemplary TSH quantification curve and corresponding calibration plot using chronoamperometry, in accordance with the embodiments of the present disclosure.

FIG. 6A through 6E illustrate exemplary TSH quantification curves and corresponding calibration plots using chronocoulometry, in accordance with the embodiments of the present disclosure.

FIGS. 7A and 7B illustrate exemplary T3 quantification using chronoamperometry, in accordance with the embodiments of the present disclosure.

FIGS. 8A and 8B illustrate exemplary T3 quantification using chronocoulometry, in accordance with the embodiments of the present disclosure.

FIGS. 9A and 9B illustrate exemplary T4 quantification using chronoamperometry, in accordance with the embodiments of the present disclosure.

FIGS. 10A and 10B illustrate exemplary T4 quantification using Electrochemical Impedance Spectroscopy (EIS), in accordance with the embodiments of the present disclosure.

### DETAILED DESCRIPTION OF THE INVENTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms are used herein. To the extent a term used in a claim is not defined below, it should be given the broadest definition persons in the pertinent art have given that term as reflected in printed publications and issued patents at the time of filing.

The present disclosure pertains to an improved electrode for an electrochemical device. In particular, the present disclosure provides an improved electrode for electrochemical device enabling detection of a biological target in a sample. Aspect of the present disclosure also provides an electrochemical device for detection of a biological target in a sample.

The present disclosure is on a premise that the inventors of the present disclosure surprisingly observed that an electrode attached with (preferably, coated with) graphene-polypyrrole based composite can significantly improve the conductivity of the electrode, which in turn can greatly improve the limit of detection (LOD) of the electrochemical device enabling quantitative detection of biological target in a sample to the tune of 0.5 fg/mL.

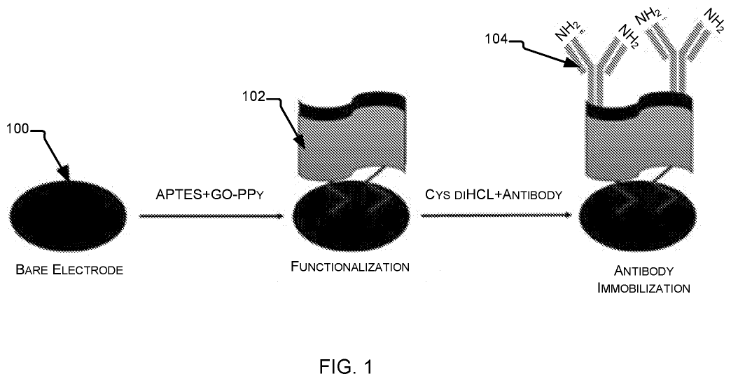

Accordingly, an aspect of the present disclosure relates to an improved electrode for an electrochemical device, the electrochemical device capable of detecting a biological target in a sample, wherein at least part of a surface of the electrode is attached with a graphene-polypyrrole based composite, and wherein the graphene-polypyrrole based composite is attached with at least one biological targeting moiety. FIG. 1 illustrates an exemplary diagram depicting an improved electrode realized in accordance with embodiments of the present disclosure. As can be seen from the figure, the electrode 100 is attached with a graphene-polypyrrole based composite 102, and the graphene-polypyrrole based composite 102 is attached with at least one biological targeting moiety 104.

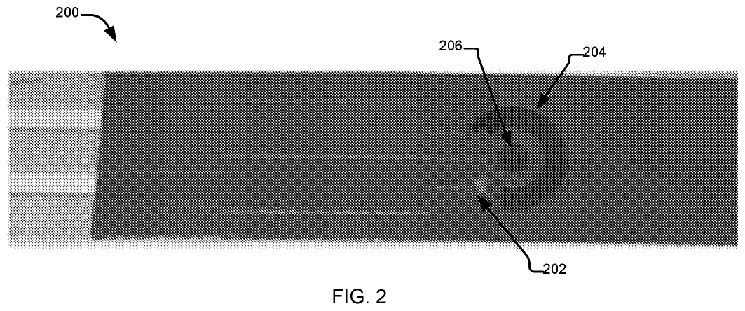

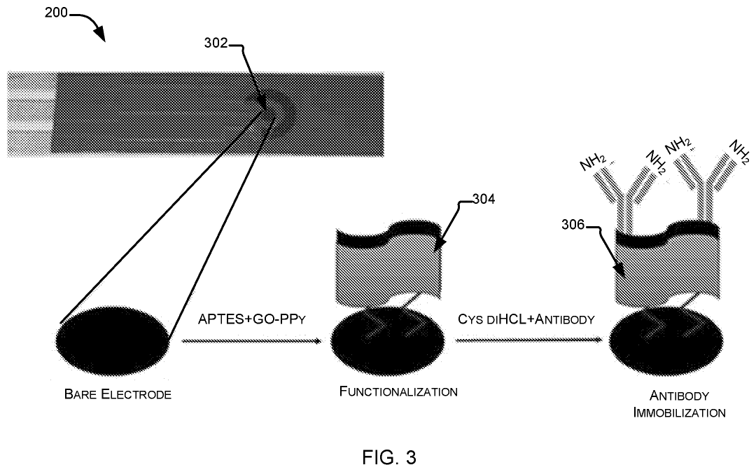

Another aspect of the present disclosure provides an electrochemical device for detection of a biological target in a sample, the electrochemical device comprising at least one electrode defining a surface, wherein at least a part of the surface of the electrode is attached with a graphene-polypyrrole based composite, and wherein the graphene-polypyrrole based composite is attached with at least one biological targeting moiety. FIG. 2 illustrates an exemplary diagram depicting an electrochemical device for detection of a biological target in a sample, in accordance with an embodiment of the present disclosure. As can be seen from the figure, the electrochemical device 200 includes a reference electrode 202, a counter electrode 204 and a working electrode 206. FIG. 3 illustrates an exemplary diagram depicting an electrochemical device for detection of a biological target in a sample including an improved electrode, realized in accordance with an embodiment of the present disclosure. As can be observed, the electrochemical device 200 includes a working electrode 302 at least a part of the surface of which is attached with a graphene-polypyrrole based composite 304, and the graphene-polypyrrole based composite 304 is attached with at least one biological targeting moiety 306.

The graphene-polypyrrole based composite, particularly, the graphene-polypyrrole based nano-composite utilized in the present disclosure can be formed using the following method: mixing pyrrole monomer with a suitable solvent in a reaction vessel by stirring at a moderate speed at an ambient temperature (about 30° C.) to prepare a first solution; and adding graphene oxide, ammonium persulfate (APS) and tetramethylethylenediamine (TEMED) to the first solution while continuously stirring the resultant reaction mixture to effect formation of the graphene-polypyrrole based nano-composite. However, it should be appreciated that any other method, as known to or appreciated by a person skilled in the pertinent art, can be utilized to realize the graphene-polypyrrole based nano-composite without departing from the scope and spirit of the present invention. In an embodiment, the graphene oxide utilized herein can be prepared by any method known to or appreciated by a person skilled in the pertinent art, preferably, the graphene oxide is prepared by modified Hummers method.

In an embodiment, the biological target is selected from any or a combination of an antibody, an antibody derivative, a hapten and an antigen. In an embodiment, the biological target is selected from any or a combination of a hormone, a protein, a polysaccharide, a lipid, a polynucleotide, and a metabolite. In an embodiment, the biological target is selected from any or a combination of thyroxine (T4), triiodothyronine (T3) and thyroid stimulating hormone (TSH). However, any other biological target, as known to or appreciated by a person skilled in the art, can be detected without departing from the scope and the spirit of the present disclosure.

In an embodiment, the electrode is made of any conductive material. A person skilled in the pertinent art is well versed with the materials that can find utility for fabrication of the electrode (particularly, the working or sensing electrode) for an electrochemical device and hence, the same is not provided in greater details for the sake of simplicity. In a preferred embodiment, the electrode is made of carbon or carbonaceous material. However, utilization of any other material for fabrication of the electrode is completely within the scope of the present disclosure.

In an embodiment, the graphene-polypyrrole based composite comprises graphene-polypyrrole based nano-composite. In an embodiment, the at least part of the surface of the electrode is coated with the graphene-polypyrrole based composite. Preferably, the whole of the surface of the electrode is coated with the graphene-polypyrrole based composite.

In an embodiment, the at least part of the surface of the electrode is functionalized with one or more amino groups capable of forming an ionic bond with the graphene-polypyrrole based composite. A person skilled in the pertinent art is well versed with the materials that can find utility for functionalization of the electrode surface with the one or more pendant amino groups and hence, not provided in greater details for the sake of simplicity. Exemplary compound that can be used for such functionalization includes 3-Aminopropyltriethoxysilane (APTES), but not limited thereto. Electrode surface functionalized with the pendant amino groups can form covalent bond with the graphene-polypyrrole based composites enabling attachment there between.

In an embodiment, the at least one biological targeting moiety comprises one or a plurality of agents capable of selectively capturing the biological target. In an embodiment, the at least one biological targeting moiety comprises one or a plurality of agents capable of non-selectively capturing the biological target. In an embodiment, the at least one biological targeting moiety comprises one or a plurality of agents capable of selectively capturing any or a combination of the antibody, the antibody derivative, the hapten and the antigen. In an embodiment, the at least one biological targeting moiety comprises one or a plurality of agents capable of non-selectively capturing any or a combination of the antibody, the antibody derivative, the hapten and the antigen. However, it is preferred that the biological targeting moiety includes one or a plurality of agents that can selectively capture the biological target as to improve the specificity and reliability of the device.

In an embodiment, the at least one biological targeting moiety is selected from any or a combination of an anti-T3 antibody, an anti-T4 antibody and an anti-TSH antibody. The electrode of the present disclosure can particularly find utility as a sensing electrode in an electrochemical device that can enable quantitative detection of any or a combination of the thyroid hormones (T3, T4 and TSH) present in a sample.

In an embodiment, the graphene-polypyrrole based composite is attached with the at least one biological targeting moiety through an amide linkage. In an embodiment, the graphene-polypyrrole based composite is functionalized with one or more amino groups capable of forming the amide linkage with Fc region of any of the anti-T3 antibody, the anti-T4 antibody and the anti-TSH antibody. A person skilled in the pertinent art is well versed with the materials that can find utility for functionalization of the graphene-polypyrrole based composites with the one or more pendant amino groups and hence, not provided in greater details for the sake of simplicity. Exemplary compound that can be used for such functionalization includes Cystamine dihydrochloride. However, utilization of any other material is completely within the scope of the present disclosure

In an embodiment, the electrochemical device exhibits the limit of detection (LOD) of 0.001 μIU/mL, 0.5 fg/mL and 0.5 fM/mL for thyroid stimulating hormone (TSH), thyroxine (T4) and triiodothyronine (T3), respectively. In an embodiment, the electrochemical device enables quantitative detection of any of a combination of the thyroid stimulating hormone (TSH), the thyroxine (T4) and the triiodothyronine (T3) within 20 minutes and more preferably, within 10 minutes.

Another aspect of the present disclosure relates to a method of fabrication of a working electrode for an electrochemical device, the method comprising the steps of: taking a working electrode; treating the working electrode with an agent capable of functionalizing at least a part of the surface of the working electrode to form a functionalized working electrode; incubating the functionalized working electrode with graphene-polypyrrole composite or nanocomposite to form a surface modified working electrode; treating the surface modified working electrode with an agent capable of functionalizing at least a part of the surface of the graphene-polypyrrole composite; and attaching at least one biological targeting moiety with the graphene-polypyrrole composite or nano-composite to realize the working electrode for the electrochemical device.

In an embodiment, the method of fabrication of a working electrode for an electrochemical device comprises the steps of: taking a working electrode; treating the working electrode with an agent capable of functionalizing at least a part of the surface of the working electrode with one or more pendant amino groups to form a functionalized working electrode; incubating the functionalized working electrode with graphene-polypyrrole composite or nanocomposite to form a surface modified working electrode; treating the surface modified working electrode with an agent capable of functionalizing at least a part of the surface of the graphene-polypyrrole composite or nanocomposite with one or more pendant amino groups; and attaching at least one biological targeting moiety with the graphene-polypyrrole composite or nano-composite to realize the working electrode for the electrochemical device.

In an embodiment, a method of fabrication of a working electrode for an electrochemical device includes the following steps: taking a working electrode; optionally, washing the working electrode with deionized (DI) water; treating the working electrode with 3-Aminopropyltriethoxysilane (APTES) to functionalize at least a part of the surface of the working electrode with one or more pendant amino groups; optionally, washing the functionalized working electrode with deionized (DI) water; incubating the functionalized working electrode with graphene-polypyrrole composite or nanocomposite; treating the surface modified working electrode with cystamine dihydrochloride to functionalize at least a part of the surface of the graphene-polypyrrole composite or nanocomposite with one or more pendant amino groups; and attaching at least one biological targeting moiety with the graphene-polypyrrole composite or nano-composite to realize the working electrode for the electrochemical device.

In an embodiment, the at least one biological targeting moiety is converted to their anionic counterpart before effecting attachment thereof with the graphene-polypyrrole composite or nano-composite. In an embodiment, the at least one biological targeting moiety includes one or a plurality of antibodies. In an embodiment, the at least one biological targeting moiety is selected from any or a combination of an anti-T3 antibody, an anti-T4 antibody and an anti-TSH antibody. In an embodiment, the one or a plurality of antibodies are treated with one or a combination of buffers having an alkaline pH, sufficient to impart negative change thereto. In an embodiment, the buffer includes bicarbonate and/or carbonate based buffer. However, use of any other buffer to serve its intended purpose as laid down in the present disclosure is completely within the scope of the present disclosure.

In an embodiment, method of fabrication of an electrode for an electrochemical device includes the following steps: fabricating an electrode using screen printing; optionally, washing the electrode with an inert fluid; treating the electrode with an agent capable of functionalizing at least a part of the surface of the electrode with one or more pendant amino groups to form a functionalized electrode; optionally, washing the functionalized electrode with an inert fluid; incubating the functionalized electrode with graphene-polypyrrole composite or nanocomposite to form a surface modified electrode; treating the surface modified electrode with an agent capable of functionalizing at least a part of the surface of the graphene-polypyrrole composite or nanocomposite with one or more pendant amino groups; and attaching at least one biological targeting moiety with the graphene-polypyrrole composite or nano-composite to realize the advantageous electrode of the present disclosure.

In an embodiment, method of fabrication of an electrode for an electrochemical device includes the following steps: fabricating a carbon based electrode using screen printing; optionally, washing the electrode with deionized (DI) water; treating the electrode with 3-Aminopropyltriethoxysilane (APTES) to functionalize at least a part of the surface of the electrode with one or more pendant amino groups; optionally, washing the functionalized electrode with deionized (DI) water; incubating the functionalized electrode with graphene-polypyrrole composite or nanocomposite; treating the surface modified electrode with cystamine dihydrochloride to functionalize at least a part of the surface of the graphene-polypyrrole composite or nanocomposite with one or more pendant amino groups; and attaching at least one biological targeting moiety with the graphene-polypyrrole composite or nano-composite to realize the advantageous electrode of the present disclosure.

In an embodiment, the at least one biological targeting moiety is converted to their anionic counterpart before effecting attachment thereof with the graphene-polypyrrole composite or nano-composite. In an embodiment, the at least one biological targeting moiety includes one or a plurality of antibodies. In an embodiment, the at least one biological targeting moiety is selected from any or a combination of an anti-T3 antibody, an anti-T4 antibody and an anti-TSH antibody. In an embodiment, the one or a plurality of antibodies are treated with one or a combination of buffers having an alkaline pH, sufficient to impart negative change thereto. In an embodiment, the buffer includes bicarbonate and/or carbonate based buffer. However, use of any other buffer to serve its intended purpose as laid down in the present disclosure is completely within the scope of the present disclosure.

Still further aspect of the present disclosure relates to a method of fabrication of an electrochemical device for detection of a biological target in a sample, the method including the steps of: fabricating a screen printed multi-electrode system out of which at least one electrode functions as a sensing (or working) electrode; optionally, washing the working electrode with an inert fluid; treating the working electrode with an agent capable of functionalizing at least a part of the surface of the working electrode to form a functionalized working electrode; optionally, washing the functionalized working electrode with an inert fluid; incubating the functionalized working electrode with graphene-polypyrrole composite or nano-composite to form a surface modified working electrode; treating the surface modified working electrode with an agent capable of functionalizing at least a part of the surface of the graphene-polypyrrole composite; and attaching at least one biological targeting moiety with the graphene-polypyrrole composite or nano-composite to realize the advantageous electrochemical device of the present disclosure.

In an embodiment, method of fabrication of an electrochemical device for detection of a biological target in a sample includes the steps of: fabricating a screen printed 3-electrode system out of which at least one electrode functions as a sensing (or working) electrode; optionally, washing the working electrode with an inert fluid; treating the working electrode with an agent capable of functionalizing at least a part of the surface of the working electrode with one or more pendant amino groups to form a functionalized working electrode; optionally, washing the functionalized working electrode with an inert fluid; incubating the functionalized working electrode with graphene-polypyrrole composite or nanocomposite to form a surface modified working electrode; treating the surface modified working electrode with an agent capable of functionalizing at least a part of the surface of the graphene-polypyrrole composite or nanocomposite with one or more pendant amino groups; and attaching at least one biological targeting moiety with the graphene-polypyrrole composite or nano-composite to realize the advantageous electrochemical device of the present disclosure.

In an embodiment, a method of fabrication of an electrochemical device for detection of a biological target in a sample, the method includes the steps of: fabricating a screen printed 3-electrode system out of which at least one electrode functions as a sensing (or working) electrode; optionally, washing the working electrode with deionized (DI) water; treating the working electrode with 3-Aminopropyltriethoxysilane (APTES) to functionalize at least a part of the surface of the working electrode with one or more pendant amino groups; optionally, washing the functionalized working electrode with deionized (DI) water; incubating the functionalized working electrode with graphene-polypyrrole composite or nanocomposite; treating the surface modified working electrode with cystamine dihydrochloride to functionalize at least a part of the surface of the graphene-polypyrrole composite or nanocomposite with one or more pendant amino groups; and attaching at least one biological targeting moiety with the graphene-polypyrrole composite or nano-composite to realize the advantageous electrochemical device of the present disclosure.

In an embodiment, the at least one biological targeting moiety is converted to their anionic counterpart before effecting attachment thereof with the graphene-polypyrrole composite or nano-composite. In an embodiment, the at least one biological targeting moiety includes one or a plurality of antibodies. In an embodiment, the at least one biological targeting moiety is selected from any or a combination of an anti-T3 antibody, an anti-T4 antibody and an anti-TSH antibody. In an embodiment, the one or a plurality of antibodies are treated with one or a combination of buffers having an alkaline pH, sufficient to impart negative change thereto. In an embodiment, the buffer includes bicarbonate and/or carbonate based buffer. However, use of any other buffer to serve its intended purpose as laid down in the present disclosure is completely within the scope of the present disclosure.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### Examples

Synthesis of Graphene-Polypyrrole (GO-PPy) Nanocomposite

Graphene oxide-Polypyrrole nanocomposite was synthesised using chemical polymerization method using graphene oxide nanosheets and pyrrole monomer. Graphene oxide nanosheets were synthesized by modified Hummers method by dissolving graphite powder (0.2 gm) and sodium nitrate (0.1 gm) in sulphuric acid (4.2 mL) while stirring it continuously for 30 minutes at ambient temperature (about 30° C.). Solution was then cooled in an ice bath for 15 minutes followed by slow addition of potassium permanganate (0.6 gm). The flask was incubated for 30 minutes in ice bath under constant stirring and then transferred to an atmosphere at 35° C. for 1 hour. This was followed by dilution of the suspension by adding 16 mL boiling water. Excess permanganate was removed by adding hydrogen peroxide (2 mL), which leads to change in color of solution from chocolate brown to yellow indicating disruption layered structure of graphene oxide. Finally, washing of the solution was done with concentrated hydrochloric acid and distilled water. This was followed by sonication for 45 minutes to ensure complete separation of graphene oxide nanosheets.

Pyrrole monomer solution was prepared by mixing 200 μL of pyrrole monomer in 5 mL water in a flask and stirred moderately over a magnetic stirrer at room temperature (about 30° C.). Then, 500 μL of graphene oxide nanosheets, along with 100 μL of 10% ammonium persulfate (APS) and 10 μL tetramethylethylenediamine (TEMED) was added to the pyrrole monomer solution while stirring continuously and final solution volume was made up to 10 mL in the flask by addition of remaining 4.19 mL of water. This was followed by ultrasonication for 10 minutes for effecting preparation of graphene oxide-polypyrrole nanocomposite.

Fabrication of Electrode

A screen printed electrode with carbon as working electrode was taken and washed with DI water. Then working electrode surface was functionalized by 5 mM 3-Aminopropyltriethoxysilane (APTES) to get NH₂ groups on the electrode surface. This functionalized electrode was then washed with DI water and incubated with graphene-polypyrrole nano-composite. This step was followed by treatment with cystamine dihydrochloride to get NH₂ groups on the surface of the graphene-polypyrrole nano-composite. Once electrode was fabricated, 0.1 μg anti-TSH antibody was immobilized over it. Before immobilization, antibody was diluted in 100 mM bicarbonate/carbonate coating buffer (pH 9.0). At this high pH, antibody gets negatively charged and COO⁻ group at the F_{c} region of antibody forms amide bond with NH₂ group present on the electrode surface. Finally, antibody immobilized electrode was blocked with 1% BSA to avoid non-specific interactions.

Parameter Optimization

Before quantification of TSH/T3/T4, various parameters like incubation temperature, time and pH of buffer, which affect the sensor response function, were optimized. Optimum time, temperature and pH of buffer for maximum immunocomplex formation were found to be 10 minutes, room temperature and 7.4, respectively.

Quantification of Thyroid Hormones

Quantification of thyroid hormones was performed by incubating different concentrations of T3/T4/TSH antigen over the working electrode under optimum time, temperature and pH conditions.

Quantification of TSH/T3/T4 in PBS pH 7.4 Sample

Different antigen concentrations were prepared in phosphate buffer saline pH 7.4. 2 μL of antigen concentration was added on the working electrode and incubated for 10 minutes at room temperature. After 10 minutes electrode was washed with PBS pH 7.4 by adding 100 μL on electrode surface using pipette 2-3 times. Finally 100 μL of 5 mM ferri/ferro cyanide in 0.01 M PBS (pH 7.4) was added on electrode surface covering all three—working, reference and auxiliary electrodes. This was followed by chronoamperometric analysis. The current thus obtained was recorded and calibration plot of current response as a function of antigen concentration was plotted. The detection limit (LOD) observed for TSH was 0.001 μIU/mL with detection range of 0.001 to 150 μIU/mL. Similarly for T3 hormone the LOD was found to be 0.5 fg/mL with detection range as 0.0005-100 pg/Ml, while LOD for T4 was found to be 0.5 fM (0.388 fg/mL) with detection range 0.0004-777 pg/mL.

Quantification of TSH/T3/T4 in Serum Sample

Entire experiment as carried out for quantification of TSH/T3/T4 in PBS pH 7.4 sample was repeated after making the concentration of TSH/T3/T4 in commercially available serum. The optimum conditions for immunocomplex formation were found to be same as that in PBS i.e. 10 minutes and room temperature. The calibration curve plotted after recording the current response curves in serum spiked with different antigen concentrations showed same LOD (TSH: 0.001 μIU/mL, T3: 0.5 fg/Ml, T4: 0.388 fg/mL) and detection range (TSH: 0.001-150 μIU/mL, T3: 0.0005-100 pg/mL, T4: 0.0004-777 pg/mL). Though the sensitivity of measurements in serum differed from that of PBS.

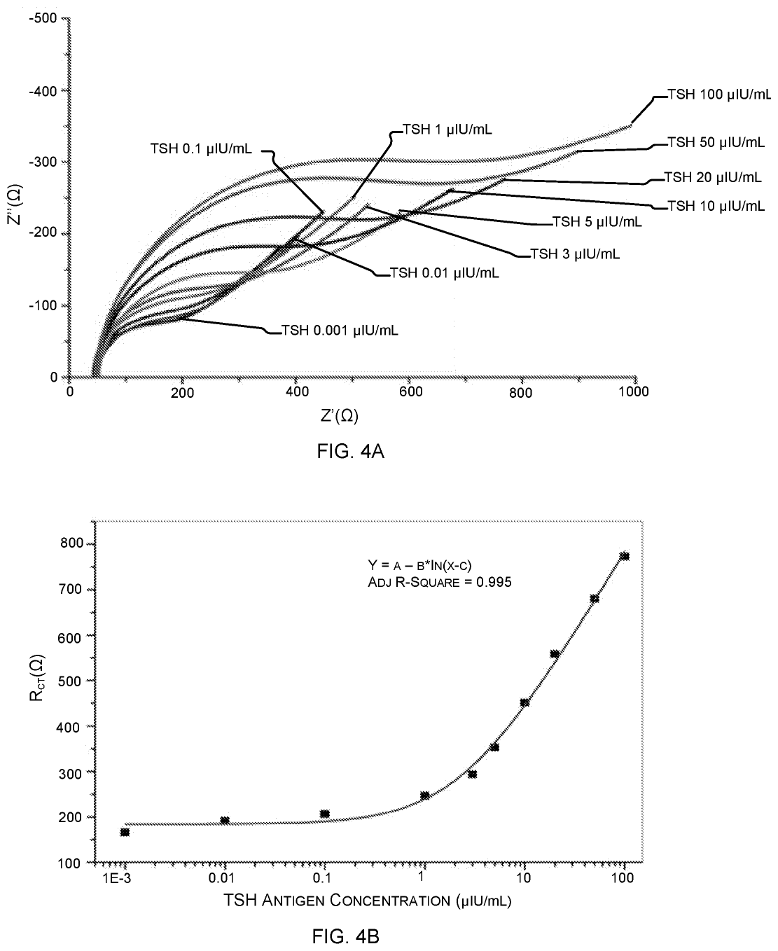

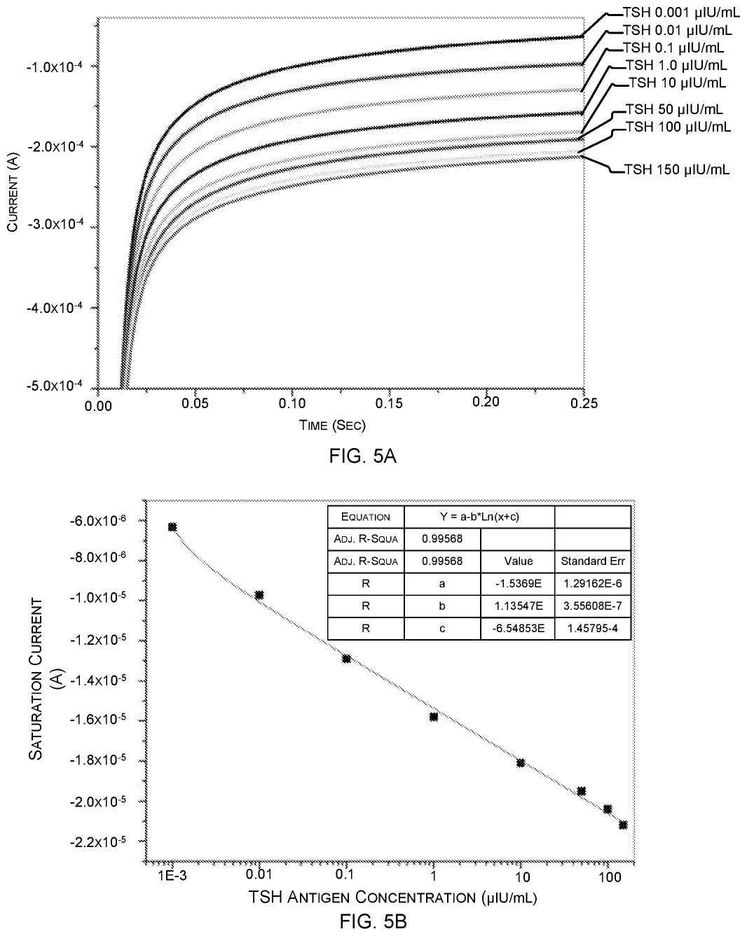

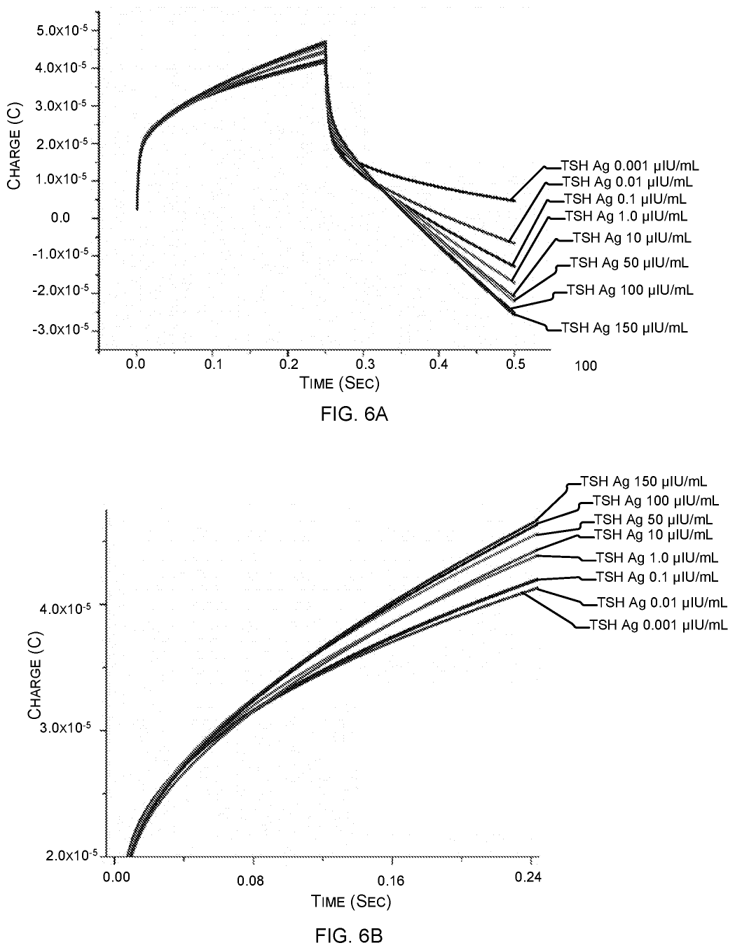

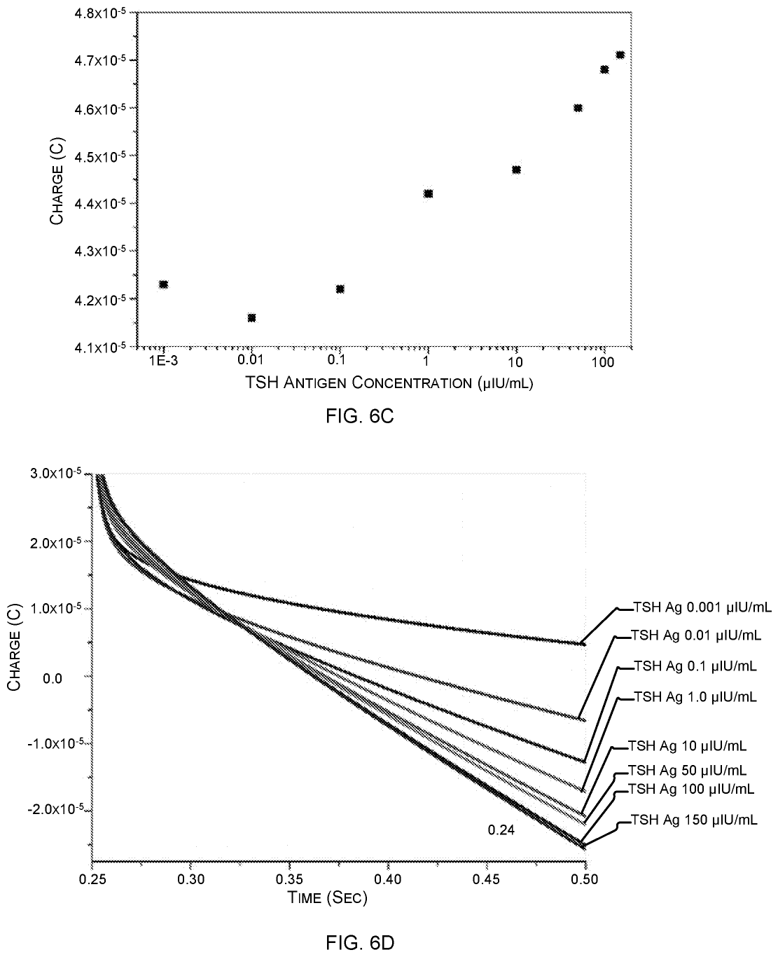

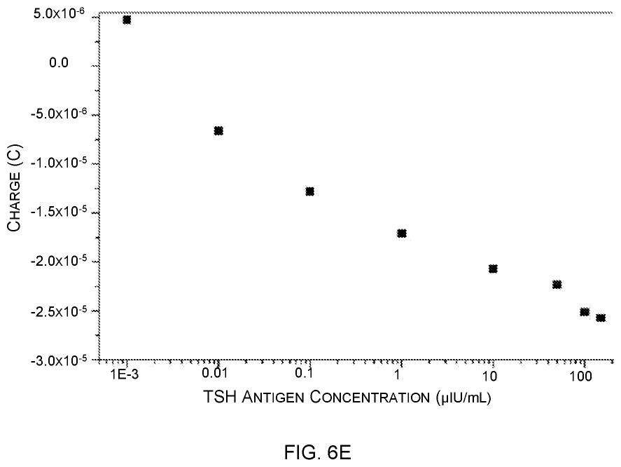

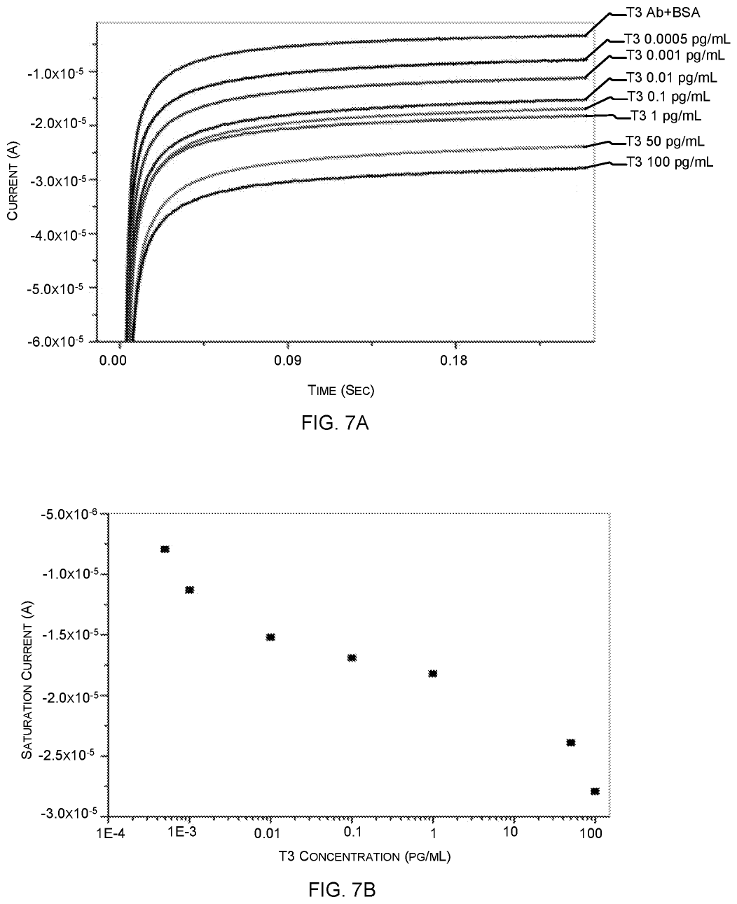

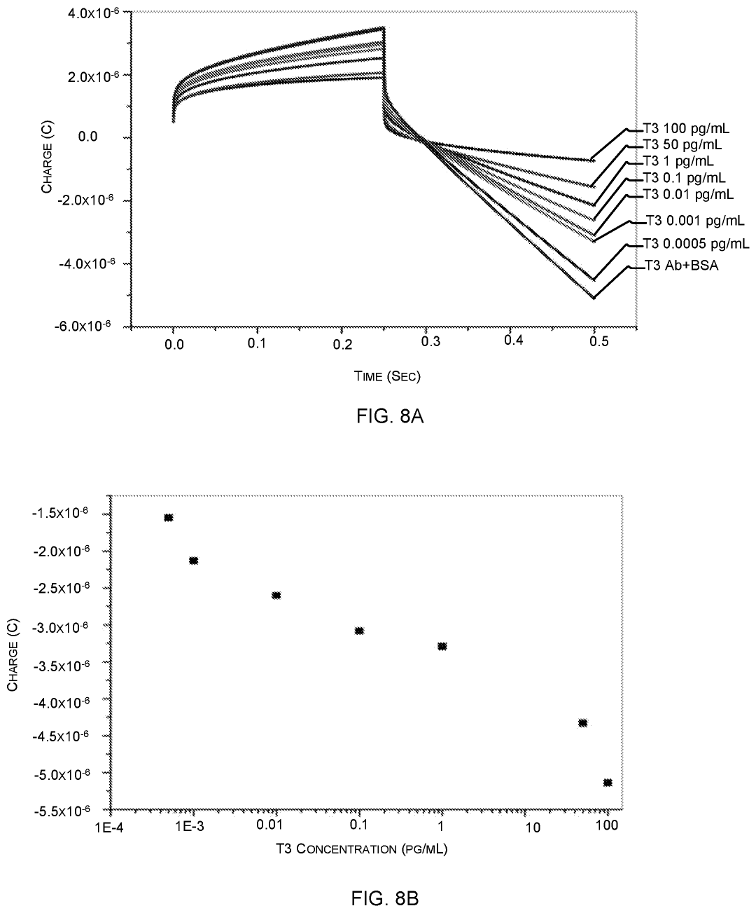

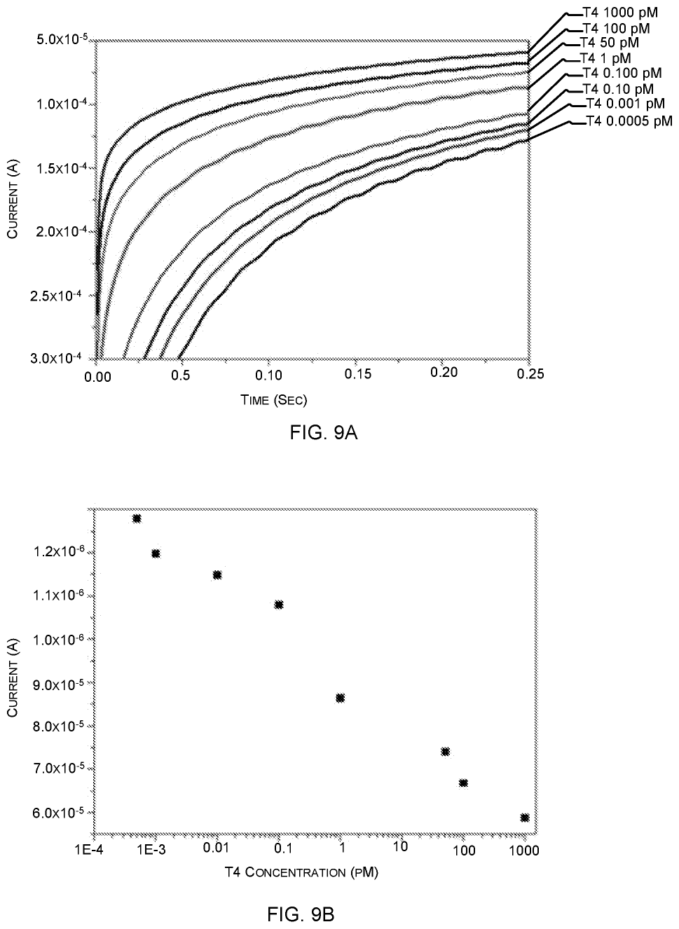

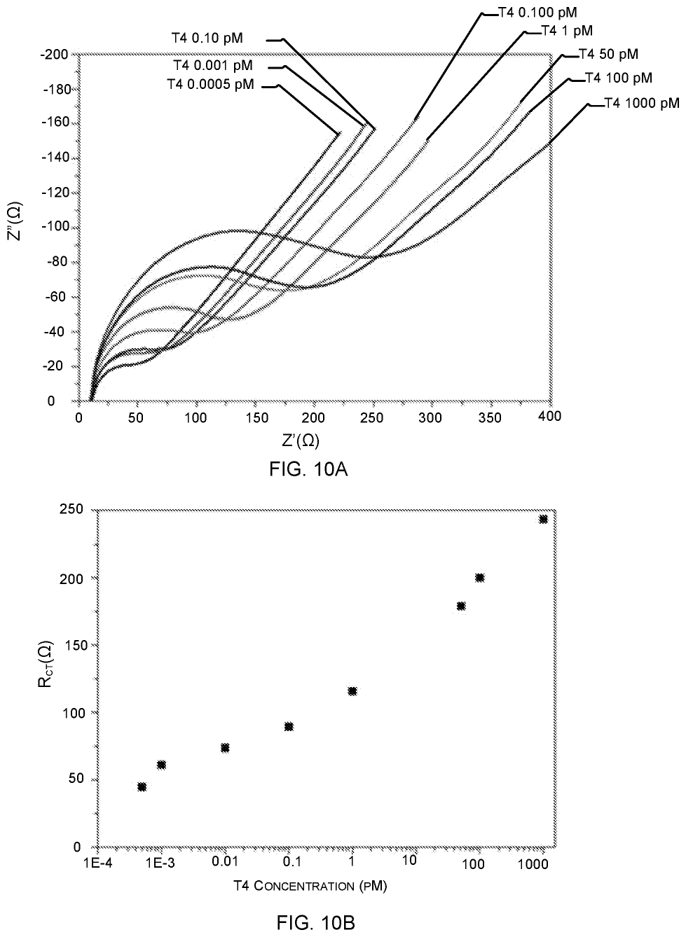

FIGS. 4A and 4B illustrate an exemplary TSH quantification curve and corresponding calibration plotusing Electrochemical Impedance Spectroscopy (EIS) in accordance with an embodiment of the present disclosure. FIGS. 5A and 5B illustrate an exemplary TSH quantification curve and corresponding calibration plot using chronoamperometry in accordance with an embodiment of the present disclosure. FIG. 6A through 6E illustrate exemplary TSH quantification curves and corresponding calibration plots using chronocoulometry in accordance with an embodiment of the present disclosure. FIGS. 7A and 7B illustrate exemplary T3 quantification using chronoamperometry in accordance with the embodiments of the present disclosure. FIGS. 8A and 8B illustrate exemplary T3 quantification using chronocoulometry in accordance with an embodiment of the present disclosure. FIGS. 9A and 9B illustrate exemplary T4 quantification using chronoamperometry in accordance with an embodiment of the present disclosure. FIGS. 10A and 10B illustrate exemplary T4 quantification using Electrochemical Impedance Spectroscopy (EIS) in accordance with an embodiment of the present disclosure.

The electrochemical device including the improved electrode of the present disclosure exhibits the LOD of 0.001 μIU/mL for TSH as opposed to LOD of 0.013 μIU/mL for CLIA based kits and 0.005 μIU/mL for electrochemiluminescence (ECL). Similarly, the electrochemical device including the improved electrode of the present disclosure exhibits the LOD of 0.5 fg/mL for T3 while that of CLIA is 0.094 ng/mL. The electrochemical device including the improved electrode of the present disclosure exhibits the LOD of 0.388 fg/mL for T4 while that of CLIA is 0.1.0 pg/mL. Based on these experiments it could be concluded that the advantageous electrode of the present disclosure and utilization thereof for fabrication of an electrochemical device for detection of a biological target in a sample greatly enhances the sensitivity and LOD values.

### Advantages of the Invention

The present disclosure provides an improved electrode for an electrochemical device.

The present disclosure provides an improved electrode for an electrochemical device capable of detecting a biological target in a sample.

The present disclosure provides an electrochemical device that can detect the biomolecule (biological target) present on a femtogram scale in the sample.

The present disclosure provides an electrochemical device for detection of thyroid hormone(s).

The present disclosure provides an electrochemical device for quantitative detection of thyroid hormone(s).

The present disclosure provides a method of fabrication of an improved electrode for an electrochemical device.

The present disclosure provides a method of fabrication of an electrochemical device for detection of a biomolecule (biological target) in the sample.

The present disclosure provides a method of quantitative detection of a biomolecule (biological target) in the sample.

The present disclosure provides a method of quantitative detection of any or a combination of thyroxine (T4), triiodothyronine (T3) and thyroid stimulating hormone (TSH) in a sample.

We claim:

1. An electrode for an electrochemical device, the electrochemical device capable of detecting a biological target in a sample, wherein at least part of a surface of the electrode is attached with a graphene-polypyrrole based composite, and wherein the graphene-polypyrrole based composite is attached with at least one biological targeting moiety.

2. The electrode as claimed in claim 1, wherein the biological target is selected from any or a combination of an antibody, an antibody derivative, a hapten, an antigen, a hormone, a protein, a polysaccharide, a lipid, a polynucleotide, a metabolite, thyroxine (T4), triiodothyronine (T3) and thyroid stimulating hormone (TSH).

3. The electrode as claimed in claim 1, wherein the graphene-polypyrrole based composite comprises graphene-polypyrrole based nano-composite.

4. The electrode as claimed in claim 1, wherein the at least part of the surface of the electrode is coated with the graphene-polypyrrole based composite.

5. The electrode as claimed in claim 1, wherein the at least part of the surface of the electrode is functionalized with one or more amino groups capable of forming covalent bond with the graphene-polypyrrole based composite.

6. The electrode as claimed in claim 1, wherein the at least one biological targeting moiety comprises one or a plurality of agents capable of selectively capturing the biological target.

7. The electrode as claimed in claim 1, wherein the at least one biological targeting moiety comprises one or a plurality of agents capable of non-selectively capturing the biological target.

8. The electrode as claimed in claim 1, wherein the at least one biological targeting moiety is selected from any or a combination of an anti-T3 antibody, an anti-T4 antibody and an anti-TSH antibody.

9. The electrode as claimed in claim 1, wherein the graphene-polypyrrole based composite is attached with the at least one biological targeting moiety through an amide linkage.

10. The electrode as claimed in claim 1, wherein the graphene-polypyrrole based composite is functionalized with one or more amino groups capable of forming the amide linkage with Fc region of any of the anti-T3 antibody, the anti-T4 antibody and the anti-TSH antibody.

11. An electrochemical device for detection of a biological target in a sample, the electrochemical device comprising at least one electrode defining a surface, wherein at least a part of the surface of the electrode is attached with a graphene-polypyrrole based composite, and wherein the graphene-polypyrrole based composite is attached with at least one biological targeting moiety.

12. The device as claimed in claim 11, wherein the biological target is selected from any or a combination of an antibody, an antibody derivative, a hapten, an antigen, a hormone, a protein, a polysaccharide, a lipid, a polynucleotide, a metabolite, thyroxine (T4), triiodothyronine (T3) and thyroid stimulating hormone (TSH).

13. The device as claimed in claim 11, wherein the at least part of the surface of the electrode is coated with the graphene-polypyrrole based composite, and wherein the graphene-polypyrrole based composite comprises graphene-polypyrrole based nano-composite.

14. The device as claimed in claim 11, wherein the at least one biological targeting moiety is selected from any or a combination of an anti-T3 antibody, an anti-T4 antibody and an anti-TSH antibody.

15. The device as claimed in claim 11, wherein the graphene-polypyrrole based composite is attached with the at least one biological targeting moiety through an amide linkage.

16. The device as claimed in claim 11, wherein the graphene-polypyrrole based composite is functionalized with one or more amino groups capable of forming the amide linkage with Fc region of any of the anti-T3 antibody, the anti-T4 antibody and the anti-TSH antibody.

17. The device as claimed in claim 11, wherein the at least one electrode is a sensing electrode.

18. The device as claimed in claim 11, wherein the electrochemical device exhibits the limit of detection (LOD) of 0.001 μIU/mL, 0.5 fg/mL and 0.5 fM for thyroid stimulating hormone (TSH), thyroxine (T4) and triiodothyronine (T3), respectively.

19. The device as claimed in claim 11, wherein the electrochemical device effects quantitative detection of any of a combination of the thyroid stimulating hormone (TSH), the thyroxine (T4) and the triiodothyronine (T3) within 20 minutes.

20. A method of fabrication of a working electrode for an electrochemical device, the method comprising the steps of:
taking a working electrode;
treating the working electrode with an agent capable of functionalizing at least a part of the surface of the working electrode to form a functionalized working electrode;
incubating the functionalized working electrode with graphene-polypyrrole composite to form a surface modified working electrode;
treating the surface modified working electrode with an agent capable of functionalizing at least a part of the surface of the graphene-polypyrrole composite; and
attaching at least one biological targeting moiety with the graphene-polypyrrole composite to realize the working electrode for the electrochemical device.
